(19) United States
(12) Patent Application Publication
(10) Pub. No.: US 2025/0288301 A1
(43) Pub. Date: Sep. 18, 2025

(54) VASO-OCCLUSIVE DEVICE AND DELIVERY ASSEMBLY

(71) Applicants: STRYKER CORPORATION, Portage, MI (US); STRYKER EUROPEAN OPERATIONS LIMITED, Carrigtwohill (IE)

(72) Inventors: Jason R. Walkingshaw, Cloyne (IE); Eamonn E. Brennan, Ballincollig (IE); Brian O'Sullivan, Blarney (IE); Alan A. Cooper, Kinsale (IE); Alan O'Donoghue, Portage, MI (US)

(73) Assignees: STRYKER CORPORATION, Portage, MI (US); STRYKER EUROPEAN OPERATIONS LIMITED, Carrigtwohill (IE)

(21) Appl. No.: 19/221,433

(22) Filed: May 28, 2025

Related U.S. Application Data
(63) Continuation of application No. PCT/US2023/081675, filed on Nov. 29, 2023.
(60) Provisional application No. 63/429,947, filed on Dec. 2, 2022.

(51) Int. Cl. A61B 17/12; A61B 17/00

(52) U.S. Cl. CPC A61B 17/1214; A61B 17/12168; A61B 2017/00477; A61B 2017/00951

(57) ABSTRACT

A vaso-occlusive treatment system includes a delivery assembly and a vaso-occlusive device detachably coupled to the delivery assembly by a delivery assembly junction. The vaso-occlusive device includes a braided portion formed out of one or more wires, the braided portion including a packed end bundle. The vaso-occlusive device also includes a coiled portion coupled to the braided portion. The vaso-occlusive device further includes an intra-device junction coupling the braided portion to the coiled portion, the intra-device junction including a stretch-resistant member spanning from the packed end bundle to the delivery assembly junction.

### RELATED APPLICATION DATA

The application is a continuation of International Patent Application No. PCT/US2023/081675, filed on Nov. 29, 2023, which claims the benefit of U.S. Provisional Patent Application Ser. No. 63/429,947, filed on Dec. 2, 2022, the entire disclosures of all of which are hereby incorporated herein by reference in their entirety into the present application.

### FIELD

The field of the disclosure generally relates to vaso-occlusive devices for establishing an embolus or vascular occlusion in a vessel of a human patient. More particularly, the disclosure relates to at least partially braided or woven vaso-occlusive devices, junctions within such devices and junctions for coupling such devices to treatment systems.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., describes a vaso-occlusive device that assumes a linear, helical primary shape when stretched for placement through the lumen of a delivery catheter, and a folded, convoluted secondary shape when released from the delivery catheter and deposited in the vasculature. All references cited herein are fully incorporated herein by reference as though set forth in full. Other examples of vaso-occlusive devices include at least partially braided or woven devices, such as those described in U.S. Pat. No. 10,321,915, issued to Murphy et al., and U.S. Pat. No. 10,893,870, issued to Wang et al.

In order to deliver the vaso-occlusive devices to a desired site in the vasculature, e.g., within an aneurysmal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 90°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” assembly or “wire” having a vaso-occlusive device coupled to its distal end is then passed through the micro-catheter, until the vaso-occlusive device pushed by the distal end of the delivery assembly is extended out of the distal end opening of the micro-catheter and into the aneurysm. Once in the aneurysm, portions of the vaso-occlusive device deform or bend to allow more efficient and complete packing. Vaso-occlusive devices that are coupled to the distal end of the delivery assembly are released or “detached” from the distal end of the delivery assembly, after extending into the aneurysm. Then, the delivery assembly is withdrawn back through the catheter. Depending on the particular needs of the patient, one or more additional vaso-occlusive devices may be pushed through the catheter and released at the same site.

One well-known way to release a vaso-occlusive device from the end of the delivery assembly is through the use of an electrolytically severable junction, which is a small exposed section or detachment zone located along a distal end portion of the delivery assembly. The detachment zone is typically made of stainless steel and is located just proximal of the vaso-occlusive device. An electrolytically severable junction is susceptible to electrolysis and disintegrates when the delivery assembly is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher completes an electrolytic detachment circuit with a return electrode, and the detachment zone disintegrates due to electrolysis. Other detachment mechanisms for releasing a vaso-occlusive device from a delivery assembly include mechanical, thermal, and hydraulic mechanisms.

In order to better frame and fill aneurysms, complex three-dimensional secondary shapes can be imparted on vaso-occlusive devices and the stiffness/flexibility of vaso-occlusive devices can be modified. However, vaso-occlusive devices continue to have performance limitations including breaking performance, shape retention and flexibility.

The proximal end of some vaso-occlusive devices is coupled to the distal end of the delivery assembly with what is known as a “main junction” of the vaso-occlusive treatment system, or a “delivery assembly junction” or simply a “delivery junction.” Another main junction design is disclosed in U.S. Pat. No. 8,202,292, issued to Kellett, which is fully incorporated herein by reference as though set forth in full. The main junction includes a flat adapter coupling a delivery wire to a vaso-occlusive device. The delivery wire has a hook or “J” shape distal end configured to be received in an aperture in the proximal end of the adapter to couple the delivery wire to the adapter. The vaso-occlusive device has windings that define openings configured to receive fingers in the distal end of the adapter to couple the vaso-occlusive device to the adapter. Consequently, the adapter facilitates coupling of the delivery wire to the vaso-occlusive device. Other main junction designs are disclosed in U.S. Pat. No. 9,480,479, issued to Chen et al., which is fully incorporated herein by reference as though set forth in full.

Some vaso-occlusive devices include one or more braided portions and one or more coiled portions. Typically, the coiled portions are attached to the distal and proximal ends of the braided portions to facilitate maneuvering the vaso-occlusive device and allowing the coiled portion to take on its secondary shape and to provide atraumatic end(s) to the vaso-occlusive device. Referring to FIG. 1, the proximal end of an example of one such vaso-occlusive device 10 is illustrated. The vaso-occlusive device 10 includes a braided portion 12 having a proximal end 14. A coil 16 having a proximal end 18 and a distal end 20 is attached to the braided portion 12. More specifically, the distal end 20 of the coil 16 is attached to the proximal end 14 of the braided portion 12. The braided portion 12 is coupled to the coil 16 by an intra-device junction 24.

Some vaso-occlusive devices also include stretch-resistant members configured to keep the coiled portions compacted or compressed to control the structural characteristics (e.g., flexibility and stiffness) of the coiled portions, which can change when coiled portions are stretched. Stretch-resistant members can form portions of both intra-device junctions and delivery junctions. In some vaso-occlusive devices, such as the device 12 shown in FIGS. 1 and 2, the stretch-resistant members are formed from one or more braid wires. FIG. 2 illustrates the braided portion 12 of the vaso-occlusive device 10 and shows two stretch-resistant members 18a, 18b formed from the braid wires of the braided portion 12. As shown in FIG. 2, the proximal end 14 of the braided portion 12 to be coupled to the coil 16 is trimmed down to only the stretch-resistant wires 18a, 18b. The stretch-resistant wires 18a, 18b are threaded from a distal end 20 of and through the coil 16 and secured at the proximal end 22a, 22b to the proximal end 18 of the coil 16. The intra-device junction 24 may also couple the distal end 20 of the coil to the distal ends 26a, 26b of the stretch resistant members 18a, 18b.

In vaso-occlusive devices such as vaso-occlusive device 10 shown in FIGS. 1 and 2, the diameters of the stretch-resistant members 18a, 18b depend on the diameters of the braid wires forming the braided portions 12. Accordingly, the structural characteristics of intra-device junctions and delivery junctions are unnecessarily dependent on characteristics of the braid wires. Consequently, vaso-occlusive devices having braided portions with fine braid wires (e.g., diameters of 0.0008 in. or 0.0009 in.) will have intra-device junctions and delivery junctions with lower tensile strength than the other structures of the elements of the vaso- occlusive devices.

Furthermore, the assembly of the components of the vaso-occlusive devices, including the braid portion, coiled portions and stretch-resistant members is complicated requiring numerous assembly steps and significant handling of the relatively fragile braid portion. For instance, the braid portion must be handled while the braid wires are trimmed at the ends to leave only the braid wires forming the stretch-resistant members, and while threading the stretch-resistant members through the coil and securing the stretch-resistant members to the coil. This results in a high percentage of rejected products and low yields, as well as a high manufacturing cost.

Accordingly, there remains a need for vaso-occlusive treatment systems having vaso-occlusive devices where the strength of the stretch-resistant member is independent of the structural characteristics of the braided portions, and methods for manufacturing which overcome the drawbacks of previous fabrication processes.

### SUMMARY

Disclosed herein are vaso-occlusive devices having a mesh portion (i.e., a braided or woven portion) and an innovative stretch resistant member independent of the mesh portion, and vaso-occlusive treatment systems utilizing such vaso-occlusive devices. According to one disclosed embodiment, a vaso-occlusive device includes a mesh portion formed out of one or more wires configured in a mesh structure. As used herein, the term “mesh” and “mesh structure” mean a structure formed of an interlaced network of wires and/or wire filament(s), including a braided structure, a woven structure or the like.

In some aspects, the mesh portion may have a primary shape (also referred to as a “delivery configuration” or “constrained configuration”) when constrained within a delivery catheter, and a secondary shape different from the primary shape (also referred to as a “deployed configuration” or “relaxed configuration”) when released from the delivery catheter to occlude a vascular defect such as an aneurysm. For instance, the primary shape may be a substantially linear shape, and the secondary shape may be a folded, 3-dimensional shape. The mesh portion has a proximal end and a distal end. As used herein, the terms “proximal” and “distal” are relative to the position of the respective elements with the device oriented as intended to be inserted into a vascular system, wherein “proximal” refers to being towards the insertion site into the vascular system (e.g., the femoral artery in patient's leg) and “distal” refers to being away from the insertion site.

The vaso-occlusive device further includes a proximal coil having a proximal end and a distal end, with the distal end coupled to the proximal end of the mesh portion. The proximal coil is configured to facilitate advancing and maneuvering the vaso-occlusive device to an insertion site (e.g., through a delivery catheter), to allow the braided portion to take on its secondary shape to fill a vascular defect and to provide a relative soft, atraumatic tip.

A first stretch resistant member is coupled to the proximal coil to restrict the proximal coil from being stretched (i.e., to restrict the coil's ability to extend, and/or to keep the coil compacted or compressed) which maintains the structural characteristics (e.g., flexibility and stiffness) of the coil. The structural characteristics of the proximal coil can be altered when stretched. The first stretch resistant member is independent of the mesh portion. As used herein, the term “independent” means that an element is not made from or comprised of another clement. In this case, the stretch resistant member is not made from or comprised of the mesh portion. For instance, the stretch resistant member is not formed of any of the wire(s) forming the mesh portion. The stretch resistant member may be a wire, rod, or other structure having a tensile strength which prevents the first stretch resistant member from being excessively stretched (e.g., opening the pitch of the winds of the coil or permanently straining the proximal coil). The first stretch resistant member extends through the proximal coil and has a proximal end coupled to the proximal end of the proximal coil and a distal end coupled to the distal end of the proximal coil.

The vaso-occlusive device also has a proximal junction physically attaching together the proximal end of the mesh portion, the distal end of the first stretch resistant member, and the distal end of the proximal coil. In other words, the proximal junction attaches the proximal end of the mesh portion to both the distal end of the first stretch resistant member and the distal end of the proximal coil, as well as attaching the proximal end of the stretch resistant member to the proximal end of the proximal coil.

In another aspect of the vaso-occlusive device, the proximal junction may consist of an adhesive. In assembling the vaso-occlusive device, the proximal end of the mesh portion, the distal end of the first stretch resistant member, and the distal end of the proximal coil arc positioned together, and the adhesive is applied to the juncture to attach them all together. In still another aspect, the proximal junction may consist of a single, integral bead of adhesive having a tapered portion. The tapered portion of the adhesive bead tapers outward as it extends distally.

Alternatively, or in addition, the proximal junction may comprise of hooking, threading, weaving, gluing, welding, soldering, sintering and/or crimping together the proximal end of the mesh portion, the distal end of the first stretch resistant member, and the distal end of the proximal coil.

In another aspect, the first stretch resistant member may comprise a single, first wire having a first end and a second end and a bend between the first end and second end. The bend forms the proximal end of the first stretch resistant member and the first end and second end of the first wire form the distal end of the first stretch resistant member. Accordingly, the bend is coupled to the proximal end proximal coil and the first and second ends of the first wire are coupled to the distal end of the proximal coil.

In yet another aspect, a coupling link may be disposed on the proximal end of the proximal coil for coupling the vaso-occlusive coil to a delivery assembly such as a delivery wire. The coupling link is also configured to couple to the proximal end of the first stretch resistant member. For example, in one embodiment, the coupling link may have a first aperture which is positioned within the proximal coil. The first wire forming the first stretch resistant member is threaded through the first aperture such that the bend in the first is disposed in the first aperture thereby coupling the proximal end of the stretch resistant member to the proximal end of the proximal coil.

In still another aspect, the first end and second end of the first wire each have a ball formed thereon to strengthen the bond between the first and second ends of the first wire and the proximal junction. The balls may be formed by micro welding the first end and second end of the first wire, or other suitable means.

In still another aspect of the vaso-occlusive device, the first end and second end of the first wire may each have a bend radially outward to strengthen the bond between the first and second ends of the first wire and the proximal junction. It can be seen that if the end portions of the first wire extend parallel or almost parallel to the longitudinal axis of the proximal coil, an axial force can more easily pull the end portions out of the proximal junction (e.g., an adhesive bead) than if the end portions are bent radially outward. In another aspect, the first and second ends have a bend radially outward of at least 20 degrees from the longitudinal axis of the proximal coil. Alternatively, the first end and second end of the first wire may be bent radially inward to obtain a similar benefit of strengthening the bond between the first and second ends of the first wire and the proximal junction. In still another aspect, the first end and or second end of the first wire may each have a hook formed thereon to strengthen the bond between the first and second ends of the first wire and the proximal junction, in much the same way as the radial bends. In another aspect, the first and second ends have a bend radially inward of at least 75 degrees from the longitudinal axis of the proximal coil.

In still another aspect, the proximal end of the mesh portion may be necked down and inserted into the distal end of the proximal coil. For instance, the mesh portion typically has a diameter greater than the proximal coil, such that the diameter of the mesh portion must be reduced to fit into the proximal coil. This feature allows an overlap of the proximal end of the mesh portion and the distal end of the proximal coil which can make it easier to effectively attach them together using the proximal junction.

In still another feature of the vaso-occlusive device, the proximal coil may have one or more spread windings between adjacent windings such that the proximal junction extends into the spread winding of the proximal coil. For instance, the adhesive or other joining means can extend through the spread winding(s) to form part of the bond between the proximal end of the mesh portion, the distal end of the first stretch resistant member, and the distal end of the proximal coil.

In other embodiments disclosed herein, the vaso-occlusive device may also include a distal coil coupled to the distal end of the mesh portion. The distal coil provides the same benefits and functions as the proximal coil, except on the distal end of the vaso-occlusive device. The distal coil has a proximal end and a distal end, with the proximal end of the distal coil coupled to the distal end of the mesh portion. A second stretch resistant member, also independent of the mesh portion, is the same or similar to the first stretch resistant member, and provides the same functions and benefits with respect to the distal coil as the first stretch resistant member provides for the proximal coil. The second stretch resistant member extends through the distal coil and has a proximal end coupled to the proximal end of the distal coil and a distal end coupled to the distal end of the distal coil. A distal junction physically attaches together the distal end of the mesh portion, the proximal end of the second stretch resistant member, and the proximal end of the distal coil. The distal junction may have all the aspects and features of the proximal junction, such consisting of an adhesive, a single, integral bead of adhesive having a tapered portion, comprising one or more of hooking, threading, weaving, gluing, welding, soldering, sintering and crimping together the distal end of the mesh portion, the proximal end of the stretch resistant member, and the proximal end of the distal coil, as described herein.

In still another aspect, the vaso-occlusive device may also have an atraumatic tip disposed on the distal end of the distal coil, to which the distal end of the stretch resistant member is coupled. For example, the atraumatic tip may comprise a ball or other shaped bead of adhesive, and the distal end of the second stretch resistant member is encapsulated in the adhesive thereby coupling the distal end of the stretch resistant member to the distal end of the distal coil.

Same or similar to the first stretch resistant member, the second stretch resistant member may comprise a second wire having a first end and a second end and a bend between the first end and second end. The bend forms the distal end of the second stretch resistant member and the first end and second end of the second resistant member form the proximal end of the second stretch resistant member.

In additional aspects, the first end and second end of the second wire may have the additional aspects and features of the first end and second of the first wire, such as the formed balls, radial bends, hooks, etc. In addition, the distal end of the mesh portion may be necked down and inserted into the proximal end of the distal coil, same or similar to the proximal end of the mesh portion.

In another aspect, the distal coil may also have one or more spread winding(s) between adjacent windings through which the distal junction extends into the spread winding of the distal coil, same or similar to the same feature of the proximal coil.

Another disclosed embodiment is directed to a vaso-occlusive treatment system utilizing any of the vaso-occlusive devices disclosed herein. In one disclosed embodiment, the vaso-occlusive treatment system includes a delivery assembly detachably coupled to the vaso-occlusive device. The delivery assembly includes a delivery catheter and a delivery or pusher device (e.g., a delivery wire). The delivery device has a linkage on a distal end of the delivery device which couples to the proximal end of the vaso-occlusive device, such as the coupling link disposed on the proximal end of the proximal coil. The linkage also includes a detachment junction for detaching the vaso-occlusive device from the delivery device. For example, the detachment junction may be an electrolytically severable (i.e., degradable) junction which disintegrates by electrolysis when electrically charged in the presence of an ionic solution (e.g., blood or other bodily fluids).

A method of using the vaso-occlusive treatment system includes advancing the delivery catheter through a patient's vasculature to a target implantation site (e.g., the location of an aneurysm to be filled by a vaso-occlusive device). Then, the delivery device with the vaso-occlusive device attached to its distal end is used to advance through the delivery catheter to the target implantation site with the vaso-occlusive device in the constrained, delivery configuration. The delivery device is used to advance the vaso-occlusive device out through the distal end opening of the delivery catheter. The vaco-occlusive device is inserted into a vascular defect (e.g., an aneurysm) at the target implantation site. As the vaso-occlusive device exits the delivery catheter and inserts into the vascular defect, the vaso-occlusive device deform and bends into its deployed configuration to fill the vascular defect. After the entire vaso-occlusive device is delivered, the detachment junction is used to detach the delivery device from the vaso-occlusive device. The delivery device may then be withdrawn through the delivery catheter. If need to fill the vascular defect, one or more additional vaso-occlusive devices may be delivered by the delivery device through the inserted delivery catheter, and inserted and released into the vascular defect in the same manner. The delivery device and delivery catheter may then be withdrawn from the patient.

Still another disclosed embodiment is directed to a method of manufacturing the vaso-occlusive devices disclosed herein. In one embodiment, a method of manufacturing the vaso-occlusive device includes providing a mesh portion. The mesh portion may have a primary shape when constrained within a delivery catheter, and a secondary shape different from the primary shape when released from the delivery catheter to occlude a vascular defect such as an aneurysm. A first stretch resistant member independent of the mesh portion, and a proximal coil are also provided. These components of the vaso-occlusive device may be provided as an assembly kit. Next, the proximal end of the mesh portion is compressed to neck down the proximal end to have a smaller diameter. This step may also be performed prior to providing the mesh portion such that the mesh portion having necked down proximal end is a part of the assembly kit.

The necked down proximal end of the mesh portion is inserted into the distal end of the proximal coil, and the first stretch resistant member is inserted through the proximal coil to position the proximal end of the first stretch resistant member adjacent to the proximal end of the proximal coil, and the distal end of the first stretch resistant member adjacent to the distal end of the proximal coil. A proximal junction is then formed to physically attach together the proximal end of the mesh portion, the distal end of the first stretch resistant member (e.g., the first and second ends of the first wire), and the distal end of the proximal coil. In one aspect, the proximal junction may include an adhesive applied to encapsulate the proximal end of the mesh portion, the distal end of the first stretch resistant member, and the distal end of the proximal coil. In another aspect, the proximal coil may have one or more spread winding(s) between adjacent windings and the proximal junction (e.g., the adhesive) extends into the spread winding of the proximal.

In other embodiments, the proximal junction may be formed by any one or more of hooking, threading, weaving, gluing, welding, soldering, sintering and crimping together the proximal end of the mesh portion, the distal end of the first stretch resistant member, and the distal end of the proximal coil.

The proximal end of the first stretch resistant member is coupled to the proximal end of the proximal coil. The proximal end of the first stretch resistant member may be coupled to the proximal end of the proximal coil prior to forming the proximal junction, such that the proximal coil with the proximal end of the first stretch resistant member coupled thereto is a part of the assembly kit. For instance, in the embodiment in which the first stretch resistant wire comprises a first wire having a first end and a second end and a bend between the first and second ends, wherein the bend forms the proximal end of the first stretch resistant member, the proximal end of the first stretch resistant member will typically be coupled to the proximal end of the proximal coil prior to forming the proximal junction. For instance, in one aspect, the vaso-occlusive device may include a coupling link disposed on the proximal end of the proximal coil. The coupling link is configured to be coupled to the distal end of the delivery device for coupling the vaso-occlusive device to the delivery assembly. As described herein, the coupling link may have a first aperture which is positioned within the proximal coil. In such case, the method of manufacture may include threading the first stretch resistant member (e.g., the first wire) through the first aperture such that the bend in the first wire is disposed in the first aperture thereby coupling the proximal end of the stretch resistant member to the proximal end of the proximal coil. This step in process can be performed before or after forming the proximal junction.

In another aspect of the method of manufacturing the vaso-occlusive device, as part of the process of attaching together the proximal end of the mesh portion, the distal end of the first stretch resistant member, and the distal end of the proximal coil, the method may also include pre-bonding the distal end of the first stretch resistant member to the distal end of the proximal coil. In one embodiment, an adhesive may be used to bond the distal end of the first stretch resistant member to the distal end of the proximal coil, such as gluing the first and second ends of the first wire to the distal end of the proximal coil. This pre-bonding process can make the step of forming the proximal junction easier and faster, which can reduce the handling of the mesh portion and rate of scrap.

The method of manufacturing may also include forming the balls, bends or hooks on the first and second ends of the first wire. This step in the process can be performed at any suitable step in the method, including without limitation, prior to starting the assembly of the components such that the kit includes the first wire with the balls, bends or hooks. Alternatively, the balls, bends of hooks on the first and second ends of the first wire may be formed after the first wire is inserted through the proximal coil but prior to forming the proximal junction.

In another aspect, the method of manufacture may also include attaching the distal coil and the second stretch resistant member to the vaso-occlusive device. The distal coil and the second stretch resistant member may be attached to the device in any of the same processes used to attach the proximal coil and first stretch resistant member. For example, the distal end of the mesh portion is compressed to neck down the proximal end to have a smaller diameter. This step may also be performed prior to providing the mesh portion such that the mesh portion having necked down distal end (and necked down proximal end) is a part of the assembly kit.

The necked down distal end of the mesh portion is inserted into the proximal end of the distal coil, and the second stretch resistant member is inserted through the distal coil to position the proximal end of the second stretch resistant member adjacent to the proximal end of the distal coil. A distal junction is then formed to physically attach together the distal end of the mesh portion, the proximal end of the second stretch resistant member (e.g., the first and second ends of the second wire), and the proximal end of the distal coil. In one aspect, the distal junction may include an adhesive applied to encapsulate the distal end of the mesh portion, the proximal end of the stretch resistant member, and the proximal end of the distal coil. In another aspect, the distal coil may have one or more spread winding(s) between adjacent windings and the distal junction (e.g., the adhesive) extends into the spread winding of the distal coil.

In other embodiments, the distal junction may be formed by any one or more of hooking, threading, weaving, gluing, welding, soldering, sintering and crimping together the distal end of the mesh portion, the proximal end of the second stretch resistant member, and the proximal end of the distal coil. The method may also include pre-bonding the proximal end of the second stretch resistant member to the proximal end of the distal coil, similar to the step of pre-bonding the distal end of the first stretch resistant member to the distal end of the proximal coil. This pre-bonding process makes the step of forming the distal junction easier and faster, which also reduces the handling of the mesh portion and rate of scrap.

The distal end of the second stretch resistant member is coupled to the distal end of the distal coil. The distal end of the second stretch resistant member may be coupled to the distal end of the distal coil prior to forming the distal junction, such that the distal coil with the distal end of the second stretch resistant member coupled thereto is a part of the assembly kit. In one aspect, the distal end of the second stretch resistant member may be coupled to the distal end of the second stretch resistant member using an adhesive, such as a ball or bead of adhesive that encapsulates the distal end of the second stretch resistant member and the distal end of the distal coil thereby coupling the distal end of the second resistant member to the distal end of the distal coil. The adhesive can be shaped and positioned to form an atraumatic tip on the distal end of the distal coil, which also constitutes the distal most end of the vaso-occlusive device.

In the case that the second stretch resistant member comprises a second wire having a first end and a second end and a bend between the first end and second end, the bend forms the distal end of the second stretch resistant member and it is coupled to the distal end of the distal coil, such as by using the ball of adhesive. The first end and second end of the second resistant member form the proximal end of the second stretch resistant member which are physically attached to the distal end of the mesh portion, and the proximal end of the distal coil by the distal junction.

### BRIEF DESCRIPTION OF THE DRAWINGS

The drawings illustrate the design and utility of various aspects of the disclosure, in which similar elements are referred to by common reference numerals. These drawings are not necessarily drawn to scale. In order to better appreciate how the above-recited and other advantages and objects are obtained, a more particular description of the disclosure will be rendered, which is illustrated in the accompanying drawings. These drawings depict only exemplary aspects of the disclosure for purposes of illustration and facilitating the below detailed description, and are not therefore to be considered limiting of its scope.

FIG. 1 is a side view of the proximal portion of a prior art vaso-occlusive device having a braided portion and stretch-resistant members coupled to a coil.

FIG. 2 is a side view of the braided portion of the vaso-occlusive device of FIG. 1 showing the stretch-resistant members formed from the wires of the braided portion.

FIG. 3 is a perspective view of a vaso-occlusive device assembly, according to one embodiment disclosed herein.

FIG. 4 is an enlarged side, cross-sectional view of the proximal portion of the vaso-occlusive device of FIG. 3 during assembly, according to one embodiment disclosed herein.

FIG. 5 is an enlarged side, cross-sectional view of the proximal portion of the vaso-occlusive device of FIG. 4 after assembly, according to one embodiment disclosed herein.

FIG. 6 is an enlarged side, cross-sectional view of the proximal portion of a vaso-occlusive device, according to another embodiment disclosed herein.

FIG. 7 is an enlarged side, cross-sectional view of the proximal portion of a vaso-occlusive device, according to another embodiment disclosed herein.

FIG. 8 is an enlarged side, cross-sectional view of the proximal portion of a vaso-occlusive device, according to another embodiment disclosed herein.

FIG. 9 is an enlarged side, cross-sectional view of the proximal portion of a vaso-occlusive device, according to another embodiment disclosed herein.

FIG. 10 is an enlarged side, cross-sectional view of the proximal portion of a vaso-occlusive device, according to another embodiment disclosed herein.

FIG. 11 is an enlarged side, cross-sectional view of the proximal portion of a vaso-occlusive device, according to another embodiment disclosed herein.

FIG. 12 is a partial cut-away, perspective view of the distal portion of a proximal coil subassembly of a vaso-occlusive device showing alternative proximal junctions, according to another embodiment disclosed herein.

FIG. 13 is a partial cut-away, perspective view of the distal portion of a proximal coil subassembly of a vaso-occlusive device showing alternative proximal junctions, according to another embodiment disclosed herein.

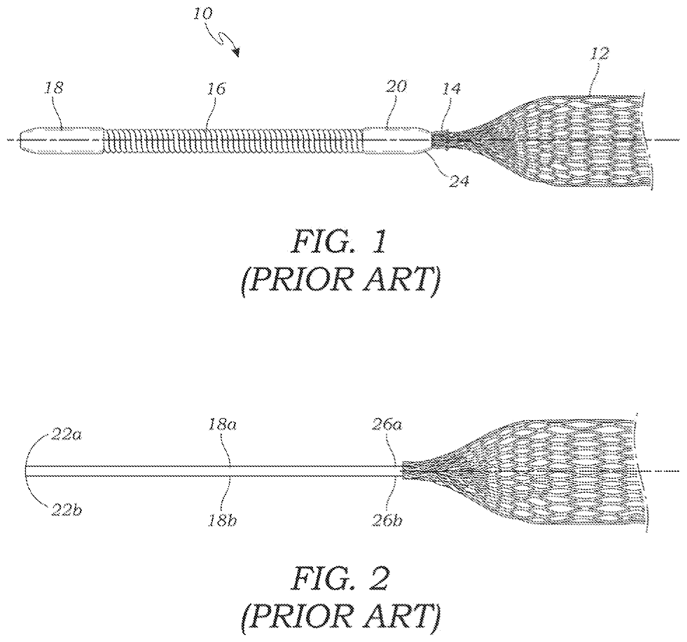

### DETAILED DESCRIPTION OF THE DISCLOSURE

This specification describes exemplary embodiments, aspects and applications of the disclosure. The disclosure, however, is not limited to these exemplary embodiments, aspects and applications or to the manner in which the exemplary embodiments, aspects and applications operate or are described herein. Further, the figures may show simplified or partial views, and the dimensions of elements in the figures may be exaggerated or otherwise not in proportion. Moreover, elements of similar structures or functions are represented by like reference numerals throughout the figures. In addition, an illustrated aspect needs not have all the features or advantages shown. A feature or an advantage described in conjunction with a particular aspect is not necessarily limited to that aspect, and can be practiced in any other aspect even if not so illustrated.

For the following defined terms, these definitions shall be applied, unless a different definition is given in the claims or elsewhere in this specification.

Where reference is made to a list of elements (e.g., elements a, b, c), such reference is intended to include any one of the listed elements by itself, any combination of less than all of the listed elements, and/or a combination of all of the listed elements.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. The term “ones” means more than one.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

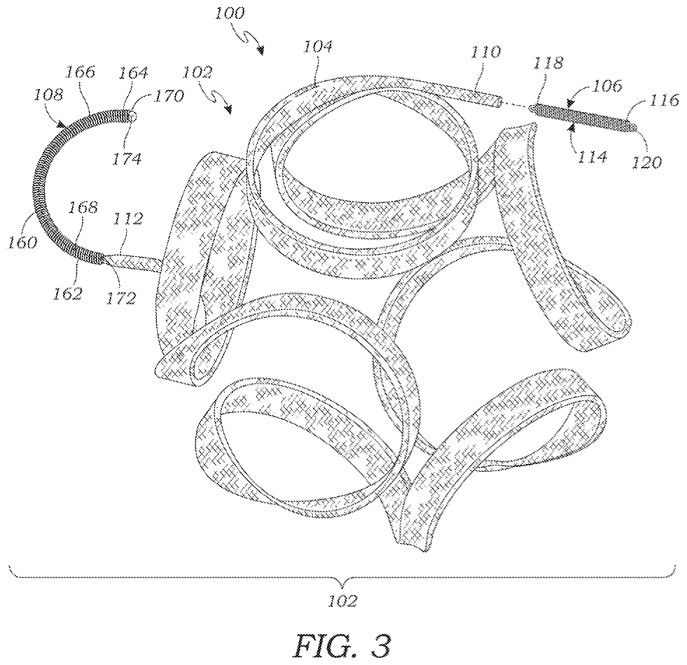

Referring to FIG. 3, an assembly 102 for a vaso-occlusive device 100 according to one disclosed embodiment is illustrated. The assembly 102 includes a mesh portion 104, a proximal coil subassembly 106 and a distal coil subassembly 108. The mesh portion 104 has a proximal end 110 and a distal end 112.

As depicted in FIG. 3, the mesh portion 104 is shown in its secondary shape which is the deployed, relaxed (unconstrained) configuration of the mesh portion 104. The mesh portion 104 may comprise a mesh formed from one or more wires interlaced into a braided structure or woven structure or the like. The wires forming the mesh portion 104 may be made of any suitable biocompatible material, including without limitation, platinum, nitinol, alloys of any of the foregoing, etc. For instance, the mesh portion 104 can be braided from 24 wires, each wire having a cross-sectional diameter of 0.001 in. The braid can be a flat braid or a round braid. According to other aspects of the disclosure, the mesh portion 104 can be braided from 16 to 32 wires, each wire having a cross-sectional diameter of 0.00075 in. to 0.0015 in. According to other aspects of the disclosure, the mesh portion 104 can be braided from more than 32 wires, each wire having a cross-sectional diameter of 0.0.00075 in. to 0.0.00125 in.

In another aspect, the wires forming the mesh portion 104 may be drawn filled tubing (DFT) available from Fort Wayne Metals in Fort Wayne, Indiana. The DFT wire includes a substantially pure Platinum (“Pt”) core at least partially surrounded by a substantially pure Nitinol (“NiTi”) external layer. As used in this application, “substantially pure” Pt includes but is not limited to 99.95% commercial purity produced in accordance with ASTM B561. The Pt core is approximately 40%-50% of the DFT wire by volume (“Pt core content”). According to other aspects of the disclosure, the DFT wires are formed by inserting a core component (a solid elongate member) into an external component (a tubular elongate member) to form a composite elongate member (e.g., a composite wire). The composite wire is repeatedly mechanically drawn and annealed by heating to increase its axial length and decrease its cross-sectional diameter. For example, the drawing and annealing process can serially reduce the diameter of a composite elongate member from 1 in. to 0.5 in., from 0.5 in. to 0.25 in., from 0.25 in. to 0.025 in., and from 0.025 in. to 0.0025 in.

While the materials (e.g., Pt and NiTi) forming various portions (e.g., core and external layer) of the DFT wire may have different stiffnesses (i.e., bending stiffness), the relative stiffnesses (i.e., bending stiffness) of the resulting portions of the DFT may not necessarily reflect the stiffnesses of the material from which they are made. For instance, even though Pt's bending modulus (i.e., stiffness) is greater than that of NiTi, Pt's bending modulus in combination with the Pt wire's diameter results in a platinum wire that is softer (i.e., less stiff) than the corresponding NiTi external layer.

The mesh portion 104 of the vaso-occlusive device 100 can be interlaced (e.g., braided or woven) on a mandrel, which can be flat or round depending on the desired final shape. The mesh portion 104 depicted in FIG. 3 has a flat, ribbon cross-section, i.e., a flat, rectangular cross-section. According to certain aspects, the mesh portion 104 can be formed with 25 wires, each wire having a cross-sectional diameter of 0.001 inches (0.0254 mm), or less than 0.001 inches (0.0254 mm). According to other aspects, the braided portion can be formed with 16 to 32 wires, each wire having a cross-section diameter of 0.0075 inches (0.191 mm) to 0.0015 inches (0.0381.

After interlacing, the mesh portion 104 can be heat set (e.g., at 500° C. to 550° C. for 1 to 10 minutes). The heat set completed mesh portion 104 forms a linear “primary shape” of the mesh portion 104. The linear primary shape is the delivery configuration or constrained configuration such as when the mesh portion 104 is constrained within a delivery catheter 186 (see FIG. 5). The heat set completed mesh portion 104 can then be wound and/or wrapped around a second mandrel (e.g., a three-dimensional mandrel) and heat set for a second time to impart the three-dimensional “secondary shape,” as depicted in FIG. 3. The NiTi external layer improves retention of the secondary shape.

While vascular implants (i.e., stents) have been formed from NiTi-DFT-Pt wires where the Pt core content is up to around 30%, NiTi-DFT-Pt wires with Pt core content around 40% to 50% have not been used to form stents. This is because stents typically require wires having higher yield strength to ultimate strength ratios (i.e., >80% of ultimate tensile strength (“UTS”)) to maintain vessel patency. On the other hand, vaso-occlusive device 100 includes DFT wires with lower yield strength to UTS ratios, which improves device characteristics including lower bending moments and improved breaking performance. According to various aspects of the disclosure, the DFT wires that form (portions of) the vaso-occlusive device 100 have yield strength to UTS ratios of <50% UTS, <60% UTS, <70% UTS and <80% UTS. Yield strength is the maximum amount of force which can be applied to a material before it begins to plastically deform. UTS is the minimum amount of force which must be applied to a material before it fails. Braiding at least a portion of a vaso-occlusive device from wires having a yield strength to UTS ratio of less than 80% UTS is critical to simultaneously achieving the characteristics of improved radiopacity, shape retention and breaking performance.

According to other aspects of the disclosure, vascular implants, including vaso-occlusive devices, are formed from NiTi-DFT-Pt wires with Pt core content of around 40% to 50%. Forming the mesh portion 104 from DFT wires with Pt core content of around 40% to 50% (“NiTi-DFT-40/50 Pt”) provides a mehs portion 104 that has (1) radiopacity throughout its entire length, (2) improved shape retention, and (3) improved breaking performance along the entire length of the braid. The Pt core provides the radiopacity for a substantial proportion of the vaso-occlusive device 110, providing radiopacity of various vaso-occlusive devices implanted into patients. The superelastic properties of the NiTi external layer contribute to the improved shape retention. The softness of the Pt contributes to the improved breaking performance, i.e., the ability of the vaso-occlusive device 100 to bend and fold to conform to the shape of body cavities. These characteristics of the mesh portion 104 results in a vaso-occlusive device 100 that is more suitable for intra-saccular embolization of vascular defects, such as aneurysms, i.e., substantially consistent visualization grey scale throughout the vaso-occlusive device 110 and optimal softness profile.

Braiding at least a portion of the mesh portion 104 from 16 to 32 DFT wires, each wire having a cross-sectional diameter of 0.00075 in. to 0.0015 in. and having a Pt content of 35% to 60% simultaneously provides the characteristics of improved radiopacity, shape retention and breaking performance. As the Pt content is increased, the effect of the increasing Pt content on decreasing flexible is reduced. Accordingly, braids woven from DFT wires having Pt content of 35% to 60% are surprisingly suitable for vaso-occlusive applications, e.g., endo-saccular applications requiring flexible devices. Such braids are especially well suited when they are woven from 24 to 32 DFT wires, each wire having a cross-sectional diameter of 0.00075 in. to 0.00125 in. According to various aspects of the disclosure, at least a portion of a vaso-occlusive device 110 is braided from 24 to 32 DFT wires, each wire having a cross-sectional diameter of 0.00075 in. to 0.00125 in. and having a Pt content of 40% to 50%.

Normally the austenite finish, or “Af” temperature of the NiTi is around 25° C., which is the temperature where the martensitic phase completes its transformation into the austenitic phase. Modifying the NiTi in the DFT such that its Af temperature is between 30° C. and 45° C. results in a softer vaso-occlusive device. Setting the Af temperature in this range achieves a desirable balance between device softness and conformability, which improves the suitability of the device for aneurysm treatment. Setting the Af of the NiTi can be accomplished by adjusting the composition of Ni in NiTi from the normal of 50% to 50.4%-50.8%. Further, the Af can be tuned with heat treatment of the NiTi. According to various aspects of the disclosure, the Af temperature is between 38° C. and 40° C.

The DFT can also include an oxide coating with a controlled thickness, which will enhance thrombogenisis (e.g., clotting) to increase vaso-occlusion within aneurysms. Preferably, the oxide coating has an average thickness between 50 nm and 500 nm. This is contrary to previous DFT braided implants (e.g., stents), from which oxide coatings are substantially removed (e.g., via electro-polishing), to generate “bright” stents. In previous DFT braided stents, oxide coating thickness is limited to less than 50 nm.

In other embodiments, instead of DFT wires, the mesh portion 104 may be braided from elongate members formed from smaller DFT wires twisted together. Each DFT wire may be made of Niti-DFT-40 Pt. Braiding elongate members (made from smaller DFT wires) instead of larger DFT wires results in a mesh portion 104 that is softer at approximately the same Pt core content. Accordingly, the mesh portion 104 according to this aspect of the disclosure (i.e., twisted elongate member braid) provides similar radiopacity with a softer braid. Such braids also provide higher surface area to promote thrombus formation, thereby enhancing aneurysm occlusion.

The proximal end 110 of the mesh portion 104 is necked down to a smaller diameter than the middle section of the mesh portion 104. The necked down proximal end 110 of the mesh portion 104 is inserted into the distal end 118 of a proximal coil 114.

The proximal coil subassembly 106 includes a proximal coil 114 having a proximal end 116 and a distal end 118. The proximal coil 114 typically has a lower bending stiffness than the mesh portion 104 so that it can facilitate advancing and maneuvering the vaso-occlusive device 100 through a delivery catheter which follows a tortuous path through a patient's vascular system to an insertion site), to facilitate the mesh portion 104 taking on its secondary shape after deployment to fill a vascular defect, and to provide a relative soft, atraumatic tip which minimizes tissue damage upon deployment of the vaso-occlusive device 100. In one aspect, the proximal coil 114 may be a coil wound from any of the types and sizes of wire described for the wires forming the mesh portion 104, or even the same type and size wire as used for the mesh portion 104. In other aspects, the proximal coil 114 may be a coil wound from a different type, size and/or material from the wire used for the mesh portion 104. The proximal coil 114 has open pitch proximal windings on the proximal end 116 of the proximal coil 114.

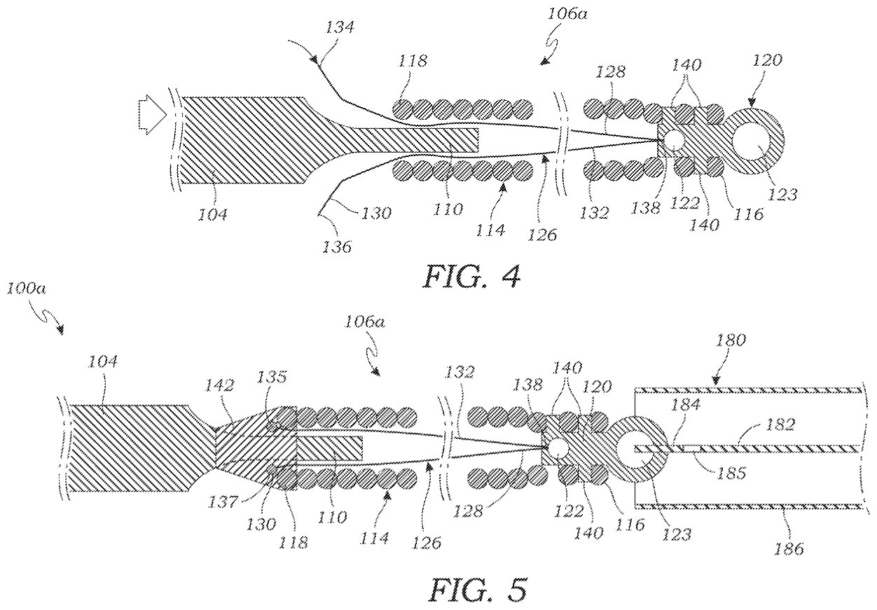

The proximal coil subassembly 106 also includes a coupling link 120 and one or more proximal stretch resistant members 126 (also referred to as “first stretch resistant member 126” or “stretch resistant member 126”). The coupling link 120 is disposed on the distal end 116 of the proximal coil 114. The coupling link 120 has a first coupler 122 for coupling the proximal end 128 of the stretch resistant member 126 to the proximal end 116 of the proximal coil 114. The coupling link 120 has a plurality of fingers 140 on the distal end of the coupling link 120 which interlace with the open pitch proximal windings on the proximal end 116 of the proximal coil 114. In the illustrated embodiments, such as the embodiment of FIGS. 4 and 5, the first coupler 122 is an aperture through the coupling link 120. The coupling link 120 is also used to couple a delivery coupler 184 on the proximal end of a delivery device 182 of a delivery assembly 180 (see e.g., FIG. 5). The coupling link 120 also has a second coupler 123 for coupling the delivery coupler 184 to the proximal end of the delivery device 182 of a delivery assembly 180. The second coupler 123 may also be an aperture through the coupling link 120.

An enlarged view of one disclosed embodiment of a proximal coil subassembly 106a is shown in FIGS. 4 and 5. FIG. 4 shows the proximal end of the vaso-occlusive device 100 prior to bonding together the mesh portion 104, the proximal stretch resistant member 126 and the proximal coil 114, and FIG. 5 shows the proximal end of the vaso-occlusive device 100 after bonding together the mesh portion 104, the stretch resistant member 126 and the proximal coil 114. The proximal stretch resistant member 126 has a proximal end 128 and a distal end 130. The proximal stretch resistant member 126 is independent of the mesh portion 104. In other words, the proximal stretch resistant member 126 is not made from or comprised of the mesh portion 104, and is not formed of any of the wire(s) forming the mesh portion 104. The proximal stretch resistant member 126 may be a wire, rod, or other structure having a tensile strength which prevents the first stretch resistant member from being excessively stretched (e.g., opening the pitch of the winds of the proximal 114 coil or permanently straining the proximal coil 114).

In the embodiment of FIGS. 4 and 5, the stretch resistant member 126 comprises a wire 132 having a first end 134, a second end 136 and a bend 138 between the first end 134 and the second end 136. The first end 134 and second end 136 form the distal end 130 of the stretch resistant member 126, and the bend 138 forms the proximal end 128 of the stretch resistant member 126. The stretch resistant member 126 extends through the proximal coil 126 and the bend 138 is threaded through the first coupler 122.

As shown in FIG. 4, the distal end 130 of the stretch resistant member 126 includes the first end 134 and second end 136 of the wire 132. The first end 134 and second end 136 are formed into a respective first ball 135 and a second ball 137 (the first ball 135 and second ball 137 now form the distal end 130 of the stretch resistant member 126), as shown in FIG. 5.

To assemble the vaso-occlusive device 100a, the necked down proximal end 110 of the mesh portion 104 is inserted into the distal end 118 of the proximal coil 114. The proximal stretch resistant member 126 is inserted through the proximal coil 114 and threaded through the first coupler 112 to couple the proximal end 128 of the stretch resistant member 126 to the proximal end 116 of the proximal coil 114 via the coupling link 120. The first ball 135 and second ball 137 (the distal end 130 of the stretch resistant member 126) are positioned adjacent to the distal end 118 of the proximal coil 114 and the necked down proximal end 110 of the mesh portion 104. A proximal junction 142 is then formed to physically attach together the proximal end 110 of the mesh portion 104, the first ball 135 and second ball 137 (the distal end 130 of the proximal stretch resistant member 126, and the distal end 118 of the proximal coil 114. As illustrated in FIG. 5, the proximal junction 142 comprises an adhesive applied to encapsulate the proximal end 110 of the mesh portion 104, the first ball 135 and second ball 137 of the proximal stretch resistant member 126, and the distal end 118 of the proximal coil 114.

As shown in FIG. 5, the vaso-occlusive device 100 is attached to a delivery assembly 180 (e.g., a delivery catheter assembly) by attaching the proximal coil subassembly 106a to the delivery assembly 180. The delivery assembly 180 includes a delivery catheter 186, a delivery device 182 (e.g., a pusher device) extending through the lumen of the delivery catheter 186 and a delivery coupler 184 on the distal end of the delivery device 182. The proximal coil subassembly 106 can be attached to the delivery device 182 by the delivery device 182 engaging the second coupler 123 of the coupling link 120. The delivery coupler 184 comprises a detachment device 185 for releasing the vaso-occlusive device 100 from the delivery device 182. In one embodiment, the detachment device 185 is an electrolytically severable junction which disintegrates via electrolysis when electrically charged in the presence of an ionic solution such as blood or other bodily fluids. Details of suitable delivery assemblies and delivery of vaso-occlusive treatment systems are described in U.S. Pat. Nos. 8,202,292, 9,480,479, 10,321,915, and 10,893,870.

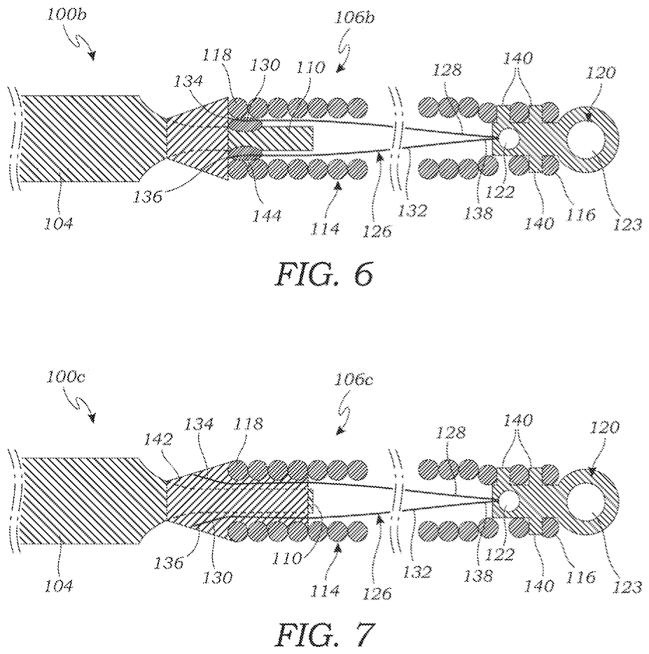

Turning now to FIGS. 6-9, several other embodiments of vaso-occlusive devices 100 having alternative embodiments of proximal coil subassemblies 106 are illustrated. The embodiments of FIGS. 6-9 have multiple step adhesive process and/or a differently formed distal end of the stretch resistant member 126 to provide better anchoring when encapsulated in the adhesive of the proximal junction 142. The vaso-occlusive device 100b having the proximal coil subassembly 106b as illustrated in FIG. 6 is substantially the same as the vaso-occlusive device 100a shown in FIG. 5, except that the vaso-occlusive device 100b has a straight first end 134 and second end 136 without balls formed on such ends, and the proximal junction 142 includes a pre glue process (also referred to as pre-bonding). The first end 134 and second end 136 of the distal end 130 of the stretch resistant member 126 terminate proximate the distal end 118 of the proximal coil 114. For example, the first end 134 and second end 135 may be trimmed to the appropriate length.

The proximal coil subassembly 106b is assembled by pre-bonding the first end 134 and second end 136 (i.e., the distal end 130 of the stretch resistant member 125) to the distal end 118 of the proximal coil 114 and/or the proximal end 110 of the mesh portion 104 inserted into the proximal coil 114. An adhesive 144 may be used to bond together the first end 134 and second end 136, the distal end 118 of the proximal coil 114, and/or the proximal end 110 of the mesh portion 104. After the adhesive 144 is allowed to cure, the proximal junction 142 is formed to physically attach together the first end 134 and second end 136 (i.e., the distal end 130 of the stretch resistant member 126) to the distal end 118 of the proximal coil 114 and/or the proximal end 110 of the mesh portion 104 inserted into the proximal coil 114, same or similar to forming the proximal junction 142 for the vaso-occlusive device 100a. This pre-bonding process strengthens the proximal junction 142 and also makes the step of forming the proximal junction 142 easier and faster, which can reduce the handling of the mesh portion and rate of scrap.

Referring to FIG. 7, a vaso-occlusive device 100c having a proximal coil subassembly 106c is substantially the same as the vaso-occlusive device 100a shown in FIG. 5, except that the vaso-occlusive device 100c has a straight first end 134 and second end 136 without balls formed on such ends, and the proximal junction 142 includes a longer bonding process allowing the adhesive to wick farther proximally along the proximal end 110 of the mesh portion 104. The first end 134 and second end 136 may also bend slightly radially outward, such as from 10-30 degrees from a longitudinal axis of the proximal coil, or greater than 20 degrees from a longitudinal axis of the proximal coil 114. The first end 134 and second end 136 of the distal end 130 of the stretch resistant member 126 extends beyond the distal end 118 of the proximal coil 114, such as extending beyond the distal end 118 by about from 1.0 to 5.0 mm. For instance, the first end 134 and second end 136 may be trimmed to the appropriate length after the desired radially outward angle is applied.

Thus, the vaso-occlusive device 100c is assembled similar to the vaso-occlusive device 100a, as described herein. Upon forming the proximal junction 142 to physically attach together the first end 134 and second end 136 (i.e., the distal end 130 of the stretch resistant member 126) to the distal end 118 of the proximal coil 114 and the proximal end 110 of the mesh portion 104 inserted into the proximal coil 114, the adhesive is applied and allowed a longer time to wick proximally along the proximal end 110 of the mesh portion 104 prior to curing. This longer process extends the area of the bond between the proximal coil 114, the first end 134 and second end 136 of the stretch resistant member 126 and the proximal end of the mesh portion 104. The results in a stronger bond, although it may also reduce the flexibility of the distal end 118 of the proximal coil 114.

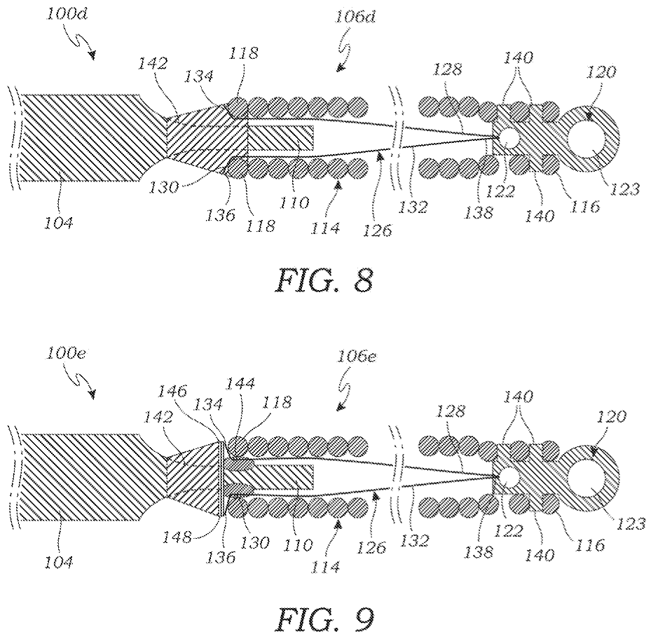

Turning now to FIG. 8, another embodiment of a vaso-occlusive coil 100d having a proximal coil subassembly 106d is illustrated. The vaso-occlusive coil 100d having the proximal coil subassembly 106d is substantially the same as the vaso-occlusive device 100a shown in FIG. 5, except that the vaso-occlusive device 100d has a stretch resistant member 126 having sharp bends at the first end 134 and second end 136. As shown in FIG. 8, the first end 134 and second end 136 of the stretch resistant member 126 have a bend radially outward at about 80 degrees. The bends in the first and second ends 134, 136 increase the strength of the bond between the stretch resistant member 126 and the proximal junction 142. Same or similar to other embodiments described herein, the first end 134 and second end 136 may be trimmed to the appropriate length after the desired radially outward angle is applied. Alternatively, the bends may be at least 20 degrees, or greater than 45 degrees, or greater than 30 degrees. The vaso-occlusive device 100d is assembled using the same process as assembling the vaso-occlusive device 100a.

Referring to FIG. 9, a vaso-occlusive device 100c having a proximal coil subassembly 106e is substantially the same as the vaso-occlusive device 100d shown in FIG. 8, except that the vaso-occlusive device 100e has the first end 134 and second end 136 each having a circumferential loop 146 and 148, respectively, wound around the proximal end 110 of the necked down mesh portion 104. The loops 146, 148 may include any suitable number of winds, such as 1, 1.5, 2, 2.5, 3 or more winds. Same or similar to other embodiments described herein, the first end 134 and second end 136 may be trimmed to the appropriate length after the desired radially outward angle is applied. The vaso-occlusive device 100e may also utilize the pre-bonding process as described for the vaso-occlusive device 100b shown in FIG. 6. Thus, the vaso-occlusive device 110d is assembled using the same process as assembling the vaso-occlusive device 100b.

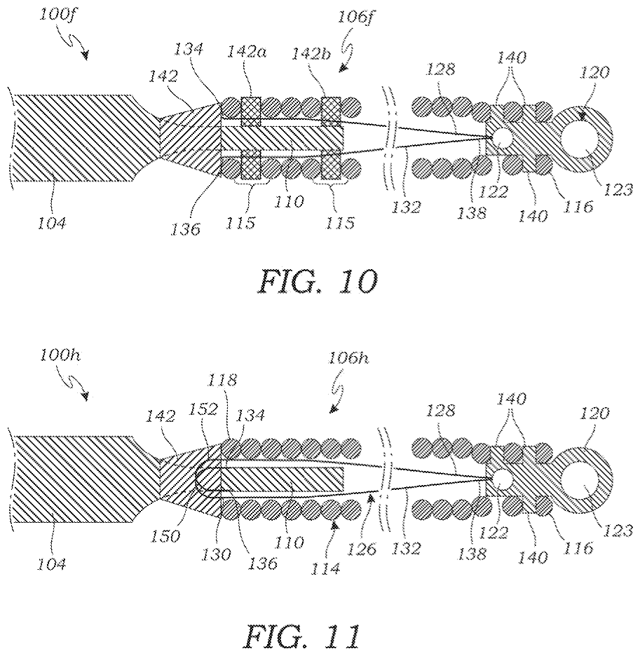

Another embodiment of a vaso-occlusive device 100f having a proximal coil subassembly 106f is illustrated in FIG. 10. The vaso-occlusive device 100f is substantially the same as the vaso-occlusive device 100b shown in FIG. 6, except that the vaso-occlusive device 100f has a proximal coil 114 having one or more spread windings 115 between adjacent windings and the proximal junction 142 extends into the spread windings of the proximal coil 114. The embodiment shown in FIG. 10 has a proximal coil 114 having two spread windings 115 toward the distal end 118 of the proximal coil 114. The adhesive, or other joining means, forming the proximal junction 142 extends through the spread windings 115 and forms part of the bond between the proximal end 110 of the mesh portion 104, the first end 134 and second end 136 of the proximal stretch resistant member 126, and the distal end 118 of the proximal coil 114. Same or similar to other embodiments described herein, the first end 134 and second end 136 may be trimmed to the appropriate length after the desired radially outward angle is applied. During assembly of the vaso-occlusive device 100f, the spread winding(s) 115 can be used to provide a visual check for the correct insertion length of the proximal end 110 of the mesh portion 104 into the proximal coil 114. For instance, the proximal end 110 of the mesh portion 104 may have a correct insertion length such that the proximal end 110 is aligned with the proximal-most spread winding 115, which can be visually confirmed by viewing through the proximal-most spread winding 115 and inserting the necked down mesh portion 104 until the proximal end is visible through the proximal-most spread winding 115. The assembly of the vaso-occlusive device 100f is substantially the same as for the vaso-occlusive device 100, and may include the pre-bonding process as described for the vaso-occlusive device 100b. In addition, the proximal junction 142 comprises adhesive inserted into the spread windings 115 such that the proximal junction 142 also includes the spread winding junctions 142a and 142b which further bond the proximal end 110 of the mesh portion 104, the first end 134 and second end 136 of the proximal stretch resistant member 126, and the distal end 118 of the proximal coil 114.

Any of the vaso-occlusive devices 100 disclosed herein, including the vaso-occlusive devices 100a-100g and 100h, can utilize the spread windings concept of the vaso-occlusive device 100f to increase the tensile strength of the vaso-occlusive device 100.

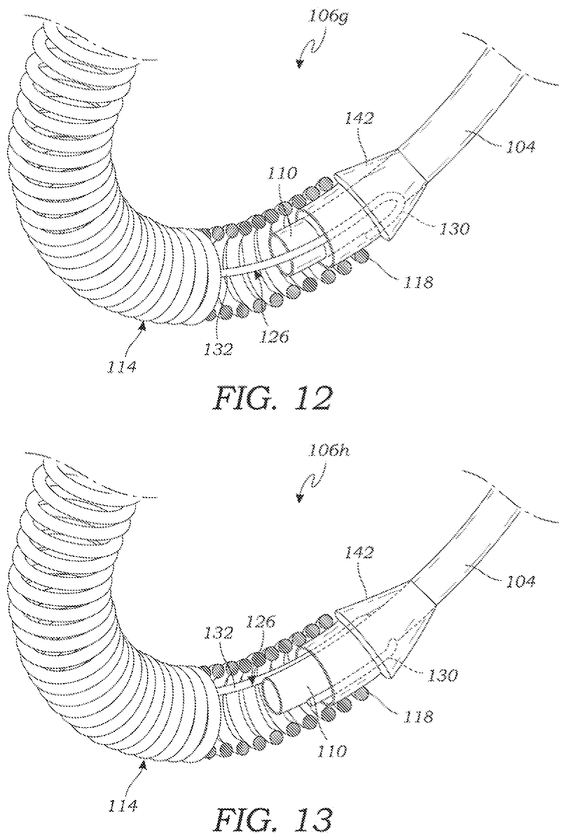

Turning now to FIG. 11, still another embodiment of a vaso-occlusive coil 100h having a proximal coil subassembly 106h is illustrated. The vaso-occlusive coil 100h having the proximal coil subassembly 106h is substantially the same as the vaso-occlusive device 100d shown in FIG. 8, except that the first end 134 and second end 136 are bent to form hooks 150 and 152, respectively. The hooks 150 and 152 increase the strength of the bond between the stretch resistant member 126 and the proximal junction 142. In an alternative embodiment, the stretch resistant member 126 may comprise a single wire attached to, and extending from, the proximal end 116 of proximal coil 114, such that the distal end 130 of the stretch resistant member 126 includes only a single hook 150 (e.g., see FIG. 12). The vaso-occlusive device 100h is assembled using the same process as assembling the vaso-occlusive device 100d.

The proximal junction 142 of the proximal coil subassembly 106 in any of the embodiments disclosed herein may use joining means other than exclusively gluing using adhesive. For example, FIGS. 15 and 16 illustrate the distal portion of additional embodiments of vaso-occlusive devices 100 having proximal junctions 142 formed by any one or more of hooking, threading, weaving, gluing, welding, soldering, sintering and crimping together the proximal end 110 of the mesh portion 104, the distal end 130 of the proximal stretch resistant member 126, and the distal end 118 of the proximal coil 114.

FIG. 12 is a partial cut-away, perspective view of the distal portion of a proximal coil subassembly 106g for a vaso-occlusive device 100 illustrating alternative embodiments for the proximal junction 142. The proximal portion of the coil subassembly 106g not shown in FIG. 12 may be the same or similar to the proximal portion of any of the coil subassemblies 106a-106f. The proximal coil subassembly 106g is similar to the proximal coil subassemblies 106 described above, except that the proximal stretch resistant member 126 does not have a bend 138 as in the other illustrated embodiments such that the wire 126 does not double back from the proximal end at the coupling link 120. Also, the proximal junction 142 is not formed exclusively by gluing. The distal end 130 of the proximal stretch resistant member 126 is formed into a hook 150 to increase the increase the strength of the bond between the stretch resistant member 126 and the proximal junction 142. The proximal junction 142 is formed by any one or more of welding, soldering, sintering and/or gluing together the proximal end 110 of the mesh portion 104, the distal end 130 (including the hook 150) of the proximal stretch resistant member 126, and the distal end 118 of the proximal coil 114.

FIG. 13 is a partial cut-away, perspective view of the distal portion of a proximal coil subassembly 106g for a vaso-occlusive device 100 illustrating additional alternative embodiments for the proximal junction 142. The proximal portion of the coil subassembly 106g not shown in FIG. 13 may be the same or similar to the proximal portion of any of the coil subassemblies 106a-106f. The proximal coil subassembly 106h is similar to the proximal coil subassemblies 106 described above, except that the proximal stretch resistant member 126 does not have a bend 138 as in the other illustrated embodiments such that the wire 126 does not double back from the proximal end at the coupling link 120. Also, the proximal junction 142 is not formed exclusively by gluing, but is instead formed by. The distal end 130 of the proximal stretch resistant member 126 is formed into a hook 150 to increase the increase the strength of the bond between the stretch resistant member 126 and the proximal junction 142. The proximal junction 142 is formed by any one or more of hooking, threading, weaving, and/or crimping the distal end 130 of the proximal stretch resistant member to the proximal end of the mesh portion 104, and welding, soldering, sintering and/or gluing together the proximal end 110 of the mesh portion 104, the distal end 130 of the proximal stretch resistant member 126, and the distal end 118 of the proximal coil 114. As can be seen in FIG. 13, the distal end 130 of the proximal stretch resistant member 126 is hooked, threaded, woven or crimped through the proximal end of the mesh portion 104. Then, the proximal junction 142 is formed by any one or more of welding, soldering, sintering and/or gluing together the proximal end 110 of the mesh portion 104, the distal end 130 of the proximal stretch resistant member 126.

Referring again to FIG. 3, each of the vaso-occlusive devices 100 disclosed herein also has a distal coil subassembly 108 attached to the distal end 112 of the mesh portion 104. The distal coil subassembly 108 includes a distal coil 160 having a proximal end 162 and a distal end 164. Same or similar to the proximal coil subassembly 106, the distal coil subassembly 108 has a distal stretch resistant member 166 (also referred to as a “second stretch resistant member 166” or simply “stretch resistant member 166”). The distal stretch resistant member 166 is also independent of the mesh portion 104, i.e., it is not made from or comprised of the mesh portion 104 or formed of any of the wire(s) forming the mesh portion 104. The distal stretch resistant member 166 may be a wire, rod, or other structure having a tensile strength which prevents the distal stretch resistant member 166 from being excessively stretched (e.g., opening the pitch of the winds of the distal coil 160 or permanently straining the distal coil 160). The distal stretch resistant member 166 has a proximal end 168 and a distal end 170. The distal stretch resistant member 166 extends through the distal coil 160. The proximal end 168 of the distal stretch resistant member 166 is attached to the proximal end 162 of the distal coil 160 and the distal end 170 of the distal stretch resistant member 166 is attached to the distal end 164 of the distal coil 160. Same or similar to the proximal end 110 of the mesh portion 104, the distal end 112 of the mesh portion 104 is necked down to insert into the proximal end 162 of the distal coil 160.

A distal junction 172 physically attaches together the proximal end 162 of the distal coil 160, the proximal end 168 of the distal stretch resistant member 166, and the distal end 172 of the mesh portion 104. Same or similar to the proximal junction 142, the distal junction 172 comprises an adhesive applied to encapsulate the proximal end 162 of the distal coil 160, the proximal end 168 of the distal stretch resistant member 166, and the distal end 172 of the mesh portion 104. For instance, the distal junction may consist of a single, integral bead of adhesive having a tapered portion. In other embodiments, the distal junction 172 may be formed by any one or more of hooking, threading, weaving, gluing, welding, soldering, sintering and crimping together the proximal end 162 of the distal coil 160, the proximal end 168 of the distal stretch resistant member 166, and the distal end 172 of the mesh portion 104, same or similar to the junctions shown in FIG. 12-13, described above.

The distal end 170 of the distal stretch resistant member 166 and the distal end 164 of the distal coil 160 may be attached together using a distal tip junction 174. The distal tip junction is formed of an adhesive, such as a ball or bead of adhesive that encapsulates the distal end 170 of the distal second stretch resistant member 166 and the distal end 164 of the distal coil 160. The adhesive forming the distal tip junction 174 is shaped and positioned to form an atraumatic tip on the distal end 164 of the distal coil 160. For instance, the atraumatic tip may be in the shape of a ball or spherical cap. The atraumatic tip constitutes the distal most end of the vaso-occlusive device 100.

Like the proximal stretch resistant member 126, the distal stretch resistant member 166 may comprise a second wire having a first end and a second end and a bend between the first end and second end. In such case, the bend forms the distal end 170 of the distal stretch resistant member 166 and is attached to the distal end 164 of the distal coil 160 by the distal tip junction 174, such as a ball of adhesive.

The vaso-occlusive devices 100 disclosed herein provide many technical advantages over previously disclosed devices. For example, the independent stretch resistant members 126, 166, allow the strength of the stretch resistant members to be independent of the characteristics of the mesh portion 104. This can result in a vaso-occlusive device having a much higher tensile strength than similarly sized devices having stretch resistant members formed from wires of the mesh portion. Furthermore, vaso-occlusive device design utilizing independent stretch resistant members 126, 166, significantly reduces the number of process steps needed to manufacture the vaso-occlusive devices. For instance, the independent stretch resistant members designs disclosed herein can reduce the manufacturing process steps by over 50% compared to devices having integrated stretch resistant member, reducing the typical number of steps from about 89 steps to about 43 steps. The simplified manufacturing considerably reduces the handling of the delicate mesh portion, thereby reducing the rate of scrap and lowering the overall manufacturing cost. Indeed, the improvements in manufacturability can decrease the overall unit production cost by about 30%.

Various aspects of the disclosure are described hereinafter with reference to the figures. It should be noted that the figures are not drawn to scale and that elements of similar structures or functions are represented by like reference numerals throughout the figures. It should also be noted that the figures are only intended to facilitate the description, and are not intended as an exhaustive description of the disclosure or as a limitation on the scope of the disclosure, which is defined only by the appended claims and their equivalents. In addition, the respective illustrated aspects need not each have all the features or advantages of aspects described herein. A feature or an advantage described in conjunction with a particular aspect of the disclosure is not necessarily limited to that aspect and can be practiced with any other aspect even if not so illustrated.

What is claimed is:

1. A vaso-occlusive device, comprising:
a mesh portion formed out of one or more wires configured in a mesh, the mesh portion having a proximal end and a distal end;
a proximal coil having a proximal end and a distal end, the distal end of the proximal coil coupled to the proximal end of the mesh portion;
a first stretch resistant member independent of the mesh portion, the first stretch resistant member extending through the proximal coil and having a proximal end coupled to the proximal end of the proximal coil and a distal end coupled to the distal end of the proximal coil; and
a proximal junction physically attaching together the proximal end of the mesh portion, the distal end of the first stretch resistant member, and the distal end of the proximal coil.

2. The vaso-occlusive device of claim 1, wherein the proximal junction consists of an adhesive.

3. The vaso-occlusive device of claim 2, wherein the proximal junction consists of a single, integral bead of adhesive having a tapered portion.

4. The vaso-occlusive device of claim 1, wherein the proximal junction comprises one or more of hooking, threading, weaving, gluing, welding, soldering, sintering and crimping together the proximal end of the mesh portion, the distal end of the first stretch resistant member, and the distal end of the proximal coil.

5. The vaso-occlusive device of claim 1, wherein:
the first stretch resistant member comprises a single, first wire having a first end and a second end and a bend between the first end and second end, and the bend forms the proximal end of the first stretch resistant member and the first end and second end of the first wire form the distal end of the first stretch resistant member.

6. The vaso-occlusive device of claim 5, further comprising:
a coupling link disposed on the proximal end of the proximal coil, the coupling link configured to couple to the proximal end of the first stretch resistant member.

7. The vaso-occlusive device of claim 6, wherein:
the coupling link has a first aperture positioned within the proximal coil; and
the first wire forming the first stretch resistant member threaded through the first aperture such that the bend is disposed in the first aperture thereby coupling the proximal end of the stretch resistant member to the proximal end of the proximal coil.

8. The vaso-occlusive device of claim 5, wherein the first end and second end of the first wire each have a ball formed thereon to strengthen a bond between the first and second ends of the first wire and the proximal junction.

9. The vaso-occlusive device of claim 8, wherein the balls are formed by micro welding the first end and second end of the first wire.

10. The vaso-occlusive device of claim 5, wherein the first end and second end of the first wire each have a bend radially outward of at least 20 degrees from a longitudinal axis of the proximal coil to strengthen a bond between the first and second ends of the first wire and the proximal junction.

11. The vaso-occlusive device of claim 5, wherein the first end and second end of the first wire each have a bend radially inward of at least 75 degrees from a longitudinal axis of the proximal coil to strengthen a bond between the first and second ends of the first wire and the proximal junction.

12. The vaso-occlusive device of claim 5, wherein the first end and second end of the first wire each have a hook formed thereon to strengthen a bond between the first and second ends of the first wire and the proximal junction.

13. The vaso-occlusive device of claim 1, wherein the proximal end of the mesh portion is necked down and inserted into the distal end of the proximal coil.

14. The vaso-occlusive device of claim 1, wherein the proximal coil has a spread winding between adjacent windings and the proximal junction extends into the spread winding of the proximal coil.

15. The vaso-occlusive device of claim 1, wherein the proximal coil has a plurality of spread windings between respective adjacent windings and the proximal junction extends into the plurality of spread windings of the proximal coil.

16. A vaso-occlusive device, comprising:
a mesh portion formed out of one or more wires configured in a mesh, the mesh portion having a proximal end and a distal end;
a proximal coil having a proximal end and a distal end, the distal end of the proximal coil coupled to the proximal end of the mesh portion;
a first stretch resistant member independent of the mesh portion, the first stretch resistant member extending through the proximal coil and having a proximal end coupled to the proximal end of the proximal coil and a distal end coupled to the distal end of the proximal coil;
a proximal junction physically attaching together the proximal end of the mesh portion, the distal end of the first stretch resistant member, and the distal end of the proximal coil;
a distal coil having a proximal end and a distal end, the proximal end of the distal coil coupled to the distal end of the mesh portion;
a second stretch resistant member independent of the mesh portion, the second stretch resistant member extending through the distal coil and having a proximal end coupled to the proximal end of the distal coil and a distal end coupled to the distal end of the distal coil; and
a distal junction physically attaching together the distal end of the mesh portion, the proximal end of the stretch resistant member, and the proximal end of the distal coil.

17. The vaso-occlusive device of claim 16, wherein the distal junction comprises one or more of hooking, threading, weaving, gluing, welding, soldering, sintering and crimping together the distal end of the mesh portion, the proximal end of the stretch resistant member, and the proximal end of the distal coil.

18. The vaso-occlusive device of claim 16, wherein:
the second stretch resistant member comprises a second wire having a first end and a second end and a bend between the first end and second end, and the bend forms the distal end of the second stretch resistant member and the first end and second end of the second resistant member form the proximal end of the second stretch resistant member.

19. The vaso-occlusive device of claim 16, wherein the first end and second end of the second wire each have a ball formed thereon to strengthen a bond between the first and second ends of the second wire to the distal junction.

20. The vaso-occlusive device of claim 16, wherein the distal coil has a spread winding between adjacent windings and the distal junction extends into the spread winding of the distal coil.
